(19) United States
(12) Patent Application Publication
(10) Pub. No.: US 2018/0133323 A1
(43) Pub. Date: May 17, 2018

(54) Optimized Fecal Microbiota Transplantation Carrier Medium

(71) Applicant: Alec D. Keith, Hilo, HI (US)

(72) Inventor: Alec D. Keith, Hilo, HI (US)

(21) Appl. No.: 15/856,745

(22) Filed: Dec. 28, 2017

(51) Int. Cl. A61K 47/10

(52) U.S. Cl. CPC A61K 47/10

(57) ABSTRACT

The present invention is a microbially inert, optimized carrier medium for fresh Fecal Microbiota Transplantation samples, intended for same-day transplantation, in which the carrier material is gastrointestinally active while remaining predominantly microbially inert. The key active agent in the present optimized collection and transplantation medium is polyethylene glycol, and in one embodiment of the invention the 1,000,000 to 10,000,000 grams per mole molecular weight of the polyethylene glycol provides a useful high viscosity to the present collection/carrier medium.

### FIELD OF THE INVENTION

The invention is an optimized carrier for fresh fecal transplant material intended for use in medical treatment purposes.

### BACKGROUND OF THE INVENTION

Gastrointestinal health can be an elusive goal even in the 21^{st} century United States and other first-world countries, and even more unattainable elsewhere. Many difficult-to-treat indications, including but not limited to initial or recurring bowel infections with Clostridium difficile, and also Ulcerative Colitis, Crohn's Disease, Irritable Bowel Syndrome, are increasingly thought to be candidates for “Fecal Microbiota Transplantion,” or FMT. However, despite certain innovation in recent years, the science of FMT is still in its infancy.

Up until now, one common way FTM was implemented was by collecting FMT material from a family member or friend. The human gastointestinal microbiota is a tissue, not an organ, and as a tissue is as or more complicated than any other human tissue. The human body contains more cells in its microbiota—by an order of magnitude—than there are cells in all the contiguous body organs. Somewhere on the order of 15,000-30,000 different bacterial species are known to inhabit the human gastrointestinal tract, with something like 500-1,500 of these living in a given patient at a given time. Of course, among these species a small subset are prevalent in each individual. Because of the complexity of the human microbiota and the genetic, nutritional, biochemical and neurological milieu in which it maintains its homeostasis, “dysbiosis,” or disruption of gastrointestinal homeostasis, can occur with or be causal to a number of syndromes or diseases involving the gut. The consequences of this dysbiosis can be severe and frequently deadly.

A near ubiquitous treatment for suspected syndromes or indications in the gastrointestinal system include oral probiotics, inevitably enteric coated in dosage form. Oral probiotics are generally accepted as wonderful, and healthful, ways to encourage positive colonization of the microbiota with desirable bacteria. Desirable bacteria are those which flourish on their own and also combat colonization of unwanted bacteria such as Clostridium difficile, by preferentially overgrowing and wiping out the trouble-making bacterial species. Many quality probiotic formulas deliver an astonishing number of live bacteria to the gut, upon enteric-coated oral administration. As a single example, certain commercial probiotic dosage forms advertise the availability of 10 billion “colony forming units” in each individual dosage form. There is little controversy that probiotics (and their culinary equivalents of yogurt, kefir and so forth) are a legitimate way to encourage and enhance the health and composition of a healthy microbiota. If anything, the enteric coated probiotics are somewhat more effective than culinary probiotics, because a predictable fraction of bacteria are killed or rendered inactive in the digestive environment of the stomach and the enteric coating protects the bacteria until they reach their final intestinal destination.

Generally speaking, as important as enteric coated probiotics are, they frequently act neither strongly nor quickly enough to restore compromised human microbiota in situations such as Clostridium difficile infection, Irritable Bowel Syndrome, Crohn's Disease and Ulcerative Colitis, to name a few. Other and arguably more drastic treatments for these conditions include monoclonal antibody active agents and surgical interventions, which have met with various degrees of success at this writing. In this context, then, FMT has garnered more and more interest as a way to restore healthy microbiota with a minimum of invasive treatment and with reduction of unwanted side effects, especially when a compatible, safe and disease-free transplant material can be obtained. Some FMT banks today make frozen and even freeze-dried FMT transplant materials available, including the option of donor-anonymous material, and often such pre-treated transplant materials have been processed enough that they are not only filtered to a particulate-free liquid but also have been purified to the point that there is no fecal smell remaining. As research continues into this pre-processed approach, however, the present inventor believes that—just as with unnecessary processing having to do with anything else regarding the gastrointestinal system—native, unprocessed transplant tissue materials predictably give a better result than frozen, freeze-dried or banked FMT materials. After all, many cryoprotectants are not native to the microbiota and can be expected to tamper with its desired natural balance. Also, up until now, the idea of optimizing a medium for use in the safe and effective collection, transport and administration of fresh FMT material has remained in its infancy, with some practitioners' defaulting to sterile normal saline or Cary Blair medium (the traditional medium for transporting stool specimens for diagnostic (pathologic) examination). Therefore, a need remains for a collection and carrier medium, for fresh FMT material, which both maintains the material during transport and which is entirely chemically compatible with the intestinal destination of the transplant.

### SUMMARY OF THE INVENTION

In order to meet this need, the present invention is a microbially inert, optimized carrier medium for fresh FMT samples, intended for same-day transplantation, in which the carrier material is gastrointestinally active while remaining predominantly microbially inert. The key active agent in the present optimized collection and transplantation medium is polyethylene glycol, as is described further below. As a shift from widespread practice, the FMT sample is collected, tested, transported and transplanted in as close as possible to its native form, which is a drastic departure from current practice. The present collection and transport medium may also be used for more traditional FMT practices that include collection, testing, filtering of the FMT to remove particulates, and administration of a solids-free, clear suspension of cells derived from the collected FMT sample. When one considers the microbiota of the body as a tissue, however, it becomes apparent (once one grasps the concept) that the microbiota is best transplanted with as little tampering as possible, in order to maximize the effect of the transplant. Why have FMT procedures nonetheless emphasized filtering of the collected material prior to administration? There are many reasons, but two predominant reasons are: 1) tissue culture experts in all fields, not just FMT, virtually always separate and purify cells in suspension as a matter of common sense wisdom and tradition; and 2) in the case of FMT, the suspension of effectively washed and filtered FMT bacteria in a clear suspension removes some of the “yuck” factor (or even horror factor) from the notion of a fecal transplant per se, given the associated taboos in Western culture and elsewhere that presume fecal matter to be useful only for diagnostic purposes, and otherwise should be considered as biological—and possibly dangerous—waste.

By using polyethylene glycol (PEG) as the key agent in an optimized FMT collection and transport medium, two key benefits are achieved:
PEG neither kills bacteria nor promotes bacterial growth, maintaining the microbial profile of the FMT material in ways that none of PEG-free nutrient medium, saline or Cary Blair medium can; and
PEG adds an intestinal motility factor to the FMT material after transplantation—usually by colonoscopic introduction—to create a dual effect of recolonization of the patient's microbiota with the healthy microbiota transplant while simultaneously discouraging lodging or retention of unwanted bacterial colonies due to decreased evacuation times encouraged by the PEG. To this end, the PEG in the present medium is present in about a five-times to one-hundred-times greater dilution than the PEG concentration of typical colonoscopy preparation solutions.

### DETAILED DESCRIPTION OF THE INVENTION

As described above, the present FMT collection and transport medium important contains polyethylene glycol (PEG), together with water and optionally any other intestinally and microbially inert excipient or diluent such as normal saline. However, in one embodiment of the invention, the present carrier and transport medium contains only PEG and sterile water, with PEG being present in the amount of about 0.25% to 5% by weight of the water. Molecular weights of the PEG can vary from around 1000 to about 10 million grams per mole; a typical molecular weight could be in the range of 2000-5000, i.e., PEG-3350. PEG is already known to be well-tolerated in the intestinal milieu, and it is a superior carrier for live FMT material because it neither curbs nor encourages bacterial growth, and therefore preserves the profile of the sample during transportation and administration. However, in a separate embodiment of the invention the PEG may have a molecular weight of about 1,000,000 to 10,000,000 g/mole (these are currently commercially available) because the high viscosity of these PEGs in aqueous solution make particularly good thick carriers for fecal transplant material, and because they are viscous carriers which completely envelope the transplant material they tend to contribute to a reduced “yuck” factor as described above. Ultimately, the PEGs of the present invention will therefore fall somewhere in the 2000 to 10,000,000 g/mole molecular weight range.

If desired, the present medium can be used as the initial collection and transport medium for FMT procedures, and the remainder of the FMT protocol can be followed without change (testing, washing, filtering, etc.). However, an insight of the present invention is that FMT material, collected and administered as a live tissue transplant, is best introduced into the patient with a minimum of disruption to the FMT sample collected—including allowing all particulate material to remain. There is a reason a healthy microbiota is healthy, and the entirety of the native constituency contributes to the overall microbiota integrity in ways we must conclude we cannot completely understand. As a practical matter, enough of the present collection and transport medium should be used to dilute the collected sample (after testing by means known in the art) to render the FMT sample of a consistency that the sample can be introduced into the patient intestines, usually by colonoscopic insertion. Other routes of administration include nasal cannula to the duodenum, enema, pessary, or where ultimately necessary enteric coated or dissolvable-or retrievable-balloon encapsulated forms introduced by mouth (as a last resort).

The idea of fecal transplant, while gaining in popularity, is still off-putting to patients and consumers, in some cases to an extreme degree. However, the dual benefit of the present invention is astonishing. First, the presence of the PEG means that the collection and transport medium will neither grow nor kill any bacterial fraction present in the collected sample, preserving the integrity of the healthy tissue chosen for transplant. Second, the relatively-low dose presence of the PEG (and if the FMT material is washed and filtered, some of the present carrier medium should be reintroduced into the final material prior to transplantation) provides the added benefit of discouraging unwanted bacteria to colonize or lodge in inflamed intestinal mucosa. In other words, the PEG provides the dual benefit of introducing healthy bacteria, within intact microbiota, that can quickly colonize and overgrow unwanted bacteria such as Clostridium difficile, while at the same time encouraging intestinal motility and passage of intestinal contents so that evacuation times shorten. The importance of this shortened evacuation time is important—even if an influx of healthy bacteria can overcome colonizations of toxin-forming gut bacteria such as Clostridium difficile, the necrotizing C. diff are still a troublesome presence in the gut and need to be expelled before they continue to inflame the mucosa and work their other damaging effects. The present invention thus provides a best-of-both-worlds in that it preserves intact a healthy FMT sample for transplantation, maximizing the opportunity for the good bacteria in the healthy transplant material to overgrow disease or syndrome-causing bacteria in the gut, while simultaneously gently increasing intestinal motility, and shortening evacuation times, in order to get the bad bacteria out of the microbiota, and out of the body altogether.

The above description has emphasized C. diff as a key indication for the present medium, and indeed it is. However, by the same action of maximized integrity of introduced transplant tissue and increased bowel evacuation “to remove troublemakers” (without increasing evacuation so much that the ability of the transplant to take hold is deterred), the present medium is useful in all FMT tissue collection and transport applications any time FMT is indicated. It should be understood that even though the invention contains PEG, the inventor does not advocate cleansing of the bowel prior to FMT, because in the case of intestinal infection, overcolonization or inflammation, cleansing procedures actually make it possible for the “bad” bacteria to take hold even more strongly, when the good bacteria that do remain in the patient's compromised microbiota are removed by the preparation procedure. The insight of the present invention is, in part, that for successful FMT transplant, the less intrusion the better with the FMT transplant material or the intestinal host destination itself, with the understanding that the low level of PEG in the present carrier medium is helpful to overall dual new colonization/enhanced evacuation.

Although the FMT in the present carrier is suitable for colonoscopic introduction without any bowel preparation in advance, simpler means of introducing FMT are also feasible—and sometimes preferable to colonoscopy. For FMT, the “scope” aspect of colonoscopy is often or typically not necessary, because any given treatment may not include any viewing or examination of the intestinal mucosa at all. Therefore, an effective FMT mechanism for the present invention is a simple introduction by enema. This enema, to introduce FMT in the present carrier, can be instituted by itself or with an immediately previous minor cleansing enema. Each patient treatment scenario will be different, because patients who are experiencing diarrhea near the time of the FMT procedure will not need any sort of pre-treatment enema, whereas in some patients a pre-treatment enema will assure comfort (avoidance of unnecessary distention) upon the introduction of the present carrier and FMT. Also, the gentle and sequential introduction of a series of time-lapsed FMT boluses can be much more comfortable for the patient—and effective for the bowel recolonization—than a single FMT procedure. Three, four or more boluses of FMT can be introduced with spacing of 5, 10, 15 or 20 minutes to maximize patient comfort and initial retention of the administered material. The present carrier medium is particularly suited to maintain the transplant tissue in the desired live-tissue status during an extended or sequential introduction of the FMT to the patient.

Although the invention has been described with particularity above, and the following example is clearly only illustrative, the invention is only to be limited insofar is set forth in the accompanying claims.

### EXAMPLE

In order to prepare enough collection medium for a single FMT collection of material according to the present invention, 100 g of sterile water is admixed, with stirring and under sterile conditions, with about 2.5 g of PEG-3350, or medical grade polyethylene having a molecular weight of 3,350. The PEG completely dissolves, yielding a clear solution. The solution thus created should be present in the collecting vessel for the fresh FMT sample and, after testing, can be used to soften the sample to the desired consistency for practical transplantation. Alternatively, because the PEG concentration of the medium is as low as it is (in this case, about 2.5% by weight) the present medium is also suitable for use both as a collection medium and as a cryoprotectant, for instances when the sample is to be frozen to preserve it for later transplantation.

I claim:

1.

2. A fecal microbiota transplant collection, transport and administration medium containing water and polyethylene glycol.

3. The medium according to claim 1 wherein said water is sterile water.

4. The medium according to claim 1 wherein said polyethylene glycol has a molecular weight between about 2000 and about 10,000,000.

5. The medium according to claim 1 wherein said polyethylene glycol has a molecular weight between about 3000 and 4000.

6. A fecal microbiota transplant collection, transport and administration medium consisting essentially of sterile water and polyethylene glycol.

7. A fecal microbiota transplant collection, transport and administration medium consisting essentially of a powdered polyethylene glycol, in unit dosage form to make a solution thereof of 0.25% to 5% by weight of water, in individual prepackaged form.

8. A fecal microbiota transplant collection, transport and administration medium consisting essentially of a polyethylene glycol carrier composed of polyethylene glycol and water, wherein said polyethylene glycol has a mean molecular weight between 1,000,000 and 10,000,000 grams per mole.
